(19) United States
(12) Patent Application Publication
(10) Pub. No.: US 2005/0238631 A1
(43) Pub. Date: Oct. 27, 2005

(54) Methods and compositions for preventing biofilm formation, reducing existing biofilms, and for reducing populations of bacteria

(76) Inventor: Steve Burwell, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 11/005,857

(22) Filed: Dec. 6, 2004

Related U.S. Application Data
(60) Provisional application No. 60/526,716, filed on Dec. 4, 2003.

(51) Int. Cl. A61K 45/00

(52) U.S. Cl. 424/93.45; 424/780

(57) ABSTRACT

Disclosed herein are compositions and methods for preventing biofilm formation, reducing existing biofilm, and/or reducing populations of pathogenic, indicator, and spoilage bacteria. In one example, disclosed are cell-free fermentates using Lactobacillus species, Pediococcus species, and/or Lactococcus species used separately, in combination, or combined with extract from Delisea pulchra marine algae.

### CROSS REFERENCE TO RELATED APPLICATIONS

This application claims the benefit of U.S. Provisional Application No. 60/526,716, filed Dec. 4, 2003. U.S. Provisional Application No. 60/526,716 is hereby incorporated herein by reference in its entirety.

### FIELD

Disclosed herein are methods and compositions for preventing biofilm formation, reducing existing biofilms, and/or for reducing populations of bacteria.

### BACKGROUND

The Centers for Disease Control (CDC) conducted an evaluation to better quantify the impact of food-borne diseases on health in the U.S. Mead, et al., compiled and analyzed information from multiple surveillance systems and other sources (Food-Related Illness and Death in the United States, Centers for Disease Control and Prevention, Atlanta, Ga., USA, 2003). The report estimated that food-borne diseases cause approximately 76 million illnesses, 325,000 hospitalizations, and 5,000 deaths in the U.S. each year. Known pathogens account for an estimated 14 million illnesses, 60,000 hospitalizations, and 1,800 deaths. Three pathogens, Salmonella, Listeria, and Toxoplasma, are responsible for 1,500 deaths each year, more than 75% of those deaths caused by known pathogens, while unknown agents account for the remaining 62 million illnesses, 265,000 hospitalizations, and 3,200 deaths. Fred R. Shank, Director of the Center for Food Safety and Applied Nutrition of the Food and Drug Administration testified before the U.S. Congress that the yearly cost of food-borne illness in the U.S. is between $7.7 and $23 billion.

In the year 2000 alone, the U.S.D.A.-Food Safety and Inspection Service reported that 18,081,829 lbs. (8,200 metric tons) of ready-to-eat meat and poultry products from 34 companies were recalled due to the presence of Listeria monocytogenes as the result of post-cooking contamination from contaminated equipment. This equated to approximately $118 million worth of product that had to be pulled from the marketplace because of this one organism in only 1 year. Further, approximately 30% of people who contract Listeria will not survive the infection. Listeria also causes spontaneous abortions. These dangers prompted the U.S.D.A.-F.S.I.S. to enact a new directive (10,240.4) that requires each company to conduct verification procedures to ensure that they are not adulterating their product with Listeria. These new directives, along with the high number of recalls, are costing the food processing industry an enormous amount of money and are resulting in extensive human suffering and a loss of corporate reputation. Because of the enormity of these problems, numerous scientific studies are being conducted to devise methods of preventing these illnesses.

A major contributing factor to contamination in the food processing industry has been identified. Pathogenic bacteria are often able to colonize food processing equipment surfaces, coolers, and freezers. When these bacteria are transferred to food processing equipment surfaces via, for example, aerosols from high pressure spraying of drains and floors, contact with contaminated workers clothes and boots, or from undercooked products, they can adhere to solid surfaces to form slimy, slippery coatings known as biofilms. In fact, Salmonella and Listeria are food-borne related pathogens that often cause infection when fully-cooked, ready-to-eat foods are contaminated after cooking because of biofilms on food processing equipment.

Wong (Biofilms in Food Processing Environments. J Dairy Sci, 81:2765-2770, 1998) reported that biofilms are able to form in the drains, belts, walls, crevices, joints, and valves in food processing plants and are a source of contamination of foods from machinery, even after cleaning and sanitizing. Wong stated that biofilms provide protection against environmental conditions that would normally destroy non-attached cells, such as cleaning and sanitizing of equipment surfaces by food production personnel. Wong concluded that even after carefully cleaning and sanitizing food processing equipment, bacterial cells still lingered on the equipment surfaces. Therefore, there is a need for methods and compositions to prevent biofilm formation, break-down or reduce existing biofilms, inhibit growth of biofilms, and/or reduce populations of bacteria, especially in the food industry. The compositions and methods disclosed herein meet this need.

### SUMMARY

In accordance with the purposes of the disclosed materials, compositions, articles, devices, and methods, as embodied and broadly described herein, the disclosed subject matter, in one aspect, relates to compositions, methods for preparing such compositions, and methods for using such compositions. In another aspect, disclosed herein are methods of contacting surfaces with such compositions. In yet another aspect, disclosed herein are methods of treating, preventing, inhibiting, and/or reducing biofilm formation and/or reducing or breaking-down existing biofilms on surfaces. In still another aspect, disclosed herein are compositions and methods for reducing the population of bacteria, for example, pathogenic, indicator, and spoilage bacteria.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF FIGURES

The accompanying figures, which are incorporated in and constitute a part of this specification, illustrate several aspects described below.

FIG. 1 is a micrograph of a biofilm. Organized channels are shown with arrows. Yeast (“y”) and bacteria (“b”) are also indicated.

FIG. 2 is a schematic of a biofilm that shows in pictorial form of how bacteria obtain food, water, and oxygen and eliminate waste in a biofilm.

FIG. 3 is a schematic of biofilm formation, arbitrarily divided into 5 stages. In stage 1, bacteria attach to a surface. In stage 2 bacteria undergo “quorum sensing” by sending signals, such as acyl-homoserine lactones. In stages 3 and 4, the biofilm grows, i.e., the glycocalyx gets larger. In stage 5, the bacteria break free from the biofilm and attach to a surface, beginning the process again.

FIG. 4 is a series of micrographs taken of Listeria at 3, 5, 7, 8, 9, 10, 11, 12, and 13 hours. The edges of the biofilm formation are indicated with arrows.

FIG. 5 is a schematic of one process disclosed herein for producing a cell-free fermentate.

FIG. 6 is a schematic of the control experiment of Example 2.

FIG. 7 is a schematic of the coating study of Example 3.

FIG. 8 is a schematic of the pre-attach study of Example 4.

FIG. 9 is a schematic of the pre-biofilm study of Example 5.

FIG. 10 is a schematic of the post-biofilm study of Example 6.

FIG. 11 is a graph of the effect of cell-free fermentate from Pediococcus acidilactici on Listeria monocytogenes (LM) colony forming units (cfu)/mL when: 1) coated onto the surface prior to exposure to LM (“coating”), 2) exposed to LM during the attachment phase of the bacterium to the coupon (“pre att”), 3) exposed to LM during biofilm formation (“pre bio”), and 4) exposed to LM after it has formed a biofilm (“post bio”).

FIG. 12 is a graph of the effect of cell-free fermentate from Lactococcus lactis subsp. lactis on Listeria monocytogenes (LM) colony forming units (cfu)/ml when: 1) coated onto the surface prior to exposure to LM (“coating”), 2) exposed to LM during the attachment phase of the bacterium to the coupon (“pre att”), 3) exposed to LM during biofilm formation (“pre bio”), and 4) exposed to LM after it has formed a biofilm (“post bio”).

FIG. 13 is a graph of the effect of cell-free fermentate from Lactobacillus acidophilus on Listeria monocytogenes (LM) colony forming units (cfu)/ml when: 1) coated onto the surface prior to exposure to LM (“coating”), 2) exposed to LM during the attachment phase of the bacterium to the coupon (“pre att”), 3) exposed to LM during biofilm formation (“pre bio”), and 4) exposed to LM after it has formed a biofilm (“post bio”).

FIG. 14 is a graph of the effect of cell-free fermentate from Lactobacillus sakei on Listeria monocytogenes (LM) colony forming units (cfu)/ml when: 1) coated onto the surface prior to exposure to LM (“coating”), 2) exposed to LM during the attachment phase of the bacterium to the coupon (“pre att”), 3) exposed to LM during biofilm formation (“pre bio”), and 4) exposed to LM after it has formed a biofilm (“post bio”).

### DETAILED DESCRIPTION

The materials, compositions, articles, devices, and methods described herein may be understood more readily by reference to the following detailed description of specific aspects of the disclosed subject matter, and methods and the Examples included therein and to the Figures and their previous and following description.

Before the present materials, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Disclosed herein are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that are possible are specifically contemplated unless specifically indicated to the contrary. Thus, if a class of components A, B, and C are disclosed as well as a class of components D, E, and F and an example of a combination composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Also, throughout this specification, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the description and claims of this specification, the word “comprise” and other forms of the word, such as “comprising” and “comprises,” means “including but not limited to,” and is not intended to exclude, for example, other additives, components, integers, or steps.

As used in the description and the appended claims, the singular forms “a,” “an,” and “the” include plural referents unless the context clearly dictates otherwise. Thus, for example, reference to “a fermentate” includes mixtures of two or more such fractions, reference to “an extract” includes mixtures of two or more such extracts, reference to “the compositions” includes mixtures of two or more such compositions, and the like.

“Optional” or “optionally” means that the subsequently described event or circumstance can or cannot occur, and that the description includes instances where the event or circumstance occurs and instances-where it does not.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By “reduce” or other forms of reduce, such as “reducing” or “reduction,” is meant lowering of an event or characteristic. It is understood that this is typically in relation to some standard or expected value, in other words it is relative, but that it is not always necessary for the standard or relative value to be referred to. For example, “reduces the population of bacteria” means lowering the amount of bacteria relative to a standard or a control.

By “inhibit” or other forms of inhibit, such as “inhibiting” or “inhibition,” is meant to hinder or restrain a particular event or characteristic or to decrease the frequency or severity of a particular event or characteristic. It is understood that this is typically in relation to some standard or expected value, in other words it is relative, but that it is not always necessary for the standard or relative value to be referred to. For example, “inhibits biofilm formation” means hindering or restraining the formation or further growth of a biofilm or decreasing the severity of biofilm formation relative to a standard or a control.

By “prevent” or other forms of prevent, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce or inhibit. As used herein, something could be reduced but not inhibited or prevented, but something that is reduced could also be inhibited or prevented. Also, something could be inhibited but not reduced or prevented, but something that is inhibited could also be reduced or prevented. Likewise, something could be prevented but not inhibited or reduced, but something that is prevented could also be inhibited or reduced. It is understood that where reduce, inhibit, or prevent are used, unless specifically indicated otherwise, the use of the other two words is also expressly disclosed. Thus, if reduces biofilm formation is disclosed, then inhibits and prevents biofilm formation are also disclosed, and the like.

By “treat” or other forms of treat, such as “treated” or “treatment,” is meant to administer a composition disclosed herein or to perform a method disclosed herein in order to reduce, inhibit, prevent, break-down, or eliminate a particular characteristic or event (e.g., biofilm formation or growth).

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Reference will now be made in detail to specific aspects of the disclosed materials, compounds, compositions, components, and methods, examples of which are illustrated in the accompanying Figures.

Disclosed herein, in one aspect, are compositions, methods for preparing such compositions, and methods for using such compositions. In another aspect, disclosed herein methods of contacting surfaces with such compositions. In yet another aspect, disclosed herein are methods of treating, preventing, inhibiting, and/or reducing biofilm formation and/or reducing or breaking-down existing biofilms on surfaces. In still another aspect, disclosed herein are compositions and methods for reducing the population of bacteria, for example, pathogenic, indicator, and spoilage bacteria.

Generally, biofilms are a collection of microorganisms surrounded by a matrix of extracellular polymers (i.e., exopolymers or glycocalyx). These extracellular polymers are typically polysaccharides, but they can contain other biopolymers as well, and they can be attached to either an inert or living surface.

Biofilms make up a sizable portion of the biomass in many environments. It is generally thought that more than 99 percent of all bacteria live in biofilm communities. In some instances, biofilm-associated forms of bacteria can outnumber their free-swimming counterparts by several orders of magnitude. Also, biofilms can contain either a single species or multiple species of bacteria.

The biofilms that can be treated (i.e., reduced, inhibited, prevented, disrupted, degraded, broken-down, eliminated, etc.) by the compositions and methods disclosed herein can be formed by Gram-positive and/or Gram-negative bacteria. Such bacteria can be pathogenic, indicator, and/or spoilage bacteria. By the compositions and methods disclosed herein, the populations of such bacteria can be treated prior to, during, or after biofilm formation. For example, a population of a Gram-positive, Gram-negative, pathogenic, indicator, and/or spoilage bacteria can be treated by the compositions and methods disclosed herein when the bacteria has not yet begun to form a biofilm, is forming a biofilm, and/or after a biofilm has formed.

The Gram-positive bacteria treatable by the compositions and methods disclosed herein can include, but are not limited to, M. tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, Propionibacterium acnes, and Enterococcus species.

The Gram-negative bacteria treatable by the compositions and methods disclosed herein can include, but are not limited to, Clostridium tetani, Clostridium perfringens, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteriacae, Brucella abortus and other Brucella species, Burkholderia cepacia, Burkholderia pseudomallei, Francisella tularensis, Bacteroides fragilis, Fusobascterium nucleatum, Provetella species, Cowdria ruminantium, Klebsiella species, and Proteus species.

The above examples of Gram-positive, Gram-negative, pathogenic, indicator, and spoilage bacteria are not intended to be limiting, but are intended to be representative of a larger population including all biofilm-associated bacteria, as well as non-Gram test responsive bacteria. Examples of other species of bacteria include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Globicatella, Gemella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella.

Biofilms can also contain other microorganisms such as, for example, parasites. Examples of parasites that can be present in biofilms, which can be treated by the compositions and methods disclosed herein, include, but are not limited to, Toxoplasma gondii, Plasmodium species such as Plasmodium falciparum, Plasmodium vivax, Plasmodium malariae, and other Plasmodium species, Trypanosoma brucei, Trypanosoma cruzi, Leishmania species such as Leishmania major, Schistosoma such as Schistosoma mansoni and other Shistosoma species, and Entamoeba histolytica.

Biofilms can further contain fungal species such as, but not limited to, Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, Alternaria alternate, and Fusarium species, which can be treated by the compositions and methods disclosed herein.

In one aspect, the biofilm can comprise one or more microorganisms chosen from Bacillus, Campylobacter, Clostridium, Enterococcus, Escherichia, Fusarium, Listeria, Proprionibacterium, Pseudomonas, Salmonella, Staphylococcus, Streptococcus, Shewanella, and Toxoplasma.

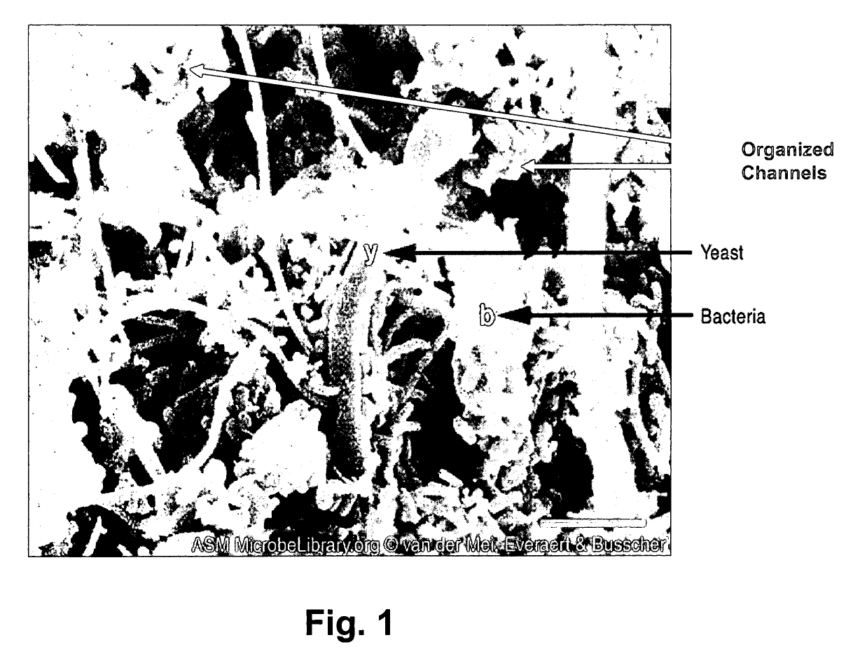

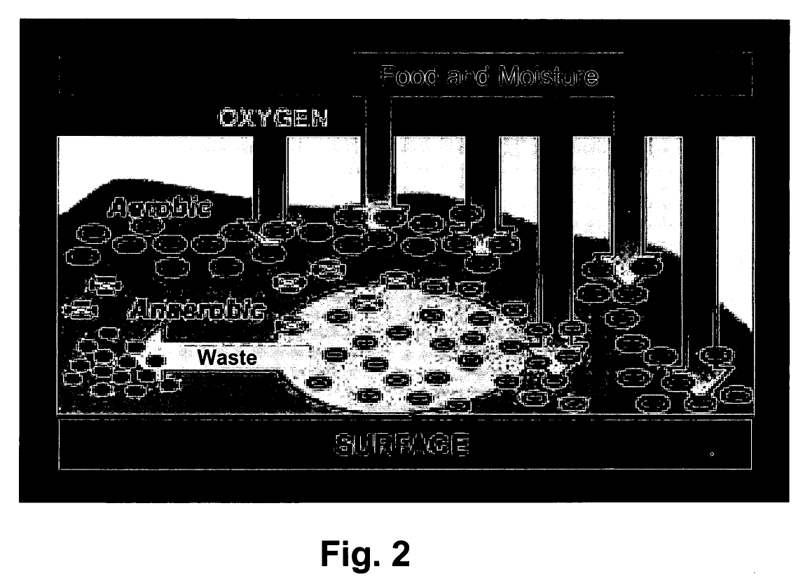

Transition from a free-swimming existence to growth in a biofilm (e.g., a biofilm on a food-processing equipment surface) can occur in response to many environmental factors, including long-term growth under conditions of nutrient deprivation or high osmolarity. The resulting biofilms can be organized into higher order structures (e.g., comprising water/nutrient channels, cellular pillars, or dense monolayers punctuated by microcolonies) that benefit the entire community (see FIGS. 1 and 2). Biofilm colonies can exhibit coordinated metabolic responses, such as spatially distinct gene expression in different regions of the biofilm that contribute to their overall fitness. Biofilms can allow bacteria to survive in hostile environments. And killing bacteria that have already formed biofilms using sanitizers can be extremely difficult.

Bacteria often form biofilms as a result of chemical signals that they receive from other bacteria. (See U.S. Pat. No. 6,559,176 to Bassler, et al., which is incorporated by reference herein for its teachings of biofilms.) Further, it has been demonstrated that bacteria can communicate with each other in order to modulate gene expression (Molina, et al., Degradation of pathogen quorum-sensing molecules by soil bacteria: a preventive and curative biological control mechanism. FEMS Microbiol Ecol, 45:71-81, 2003, which is incorporated by reference herein for its teachings of quorum sensing). This phenomenon is termed “quorum sensing” and is recognized as a general mechanism for gene regulation in many bacteria (e.g., Gram-negative and Gram-positive) that allows them to perform in unison such activities as biofilm formation, bioluminescence, swarming, production of proteolytic enzymes, synthesis of antibiotics, development of genetic competence, plasmid conjugal transfer, and spoliation (U.S. Pat. No. 6,559,176 to Bassler, et al., which is incorporated by reference herein for its teachings of quorum sensing). Quorum sensing bacteria synthesize, release, and respond to signaling molecules called autoinducers as a means of controlling gene expression as cell densities change. Many of these bacteria (e.g., Listeria, Salmonella, Escherichia, and Pseudomonas) use acyl-homoserine lactone signals for quorum sensing. (See Bassler and Silverman, Two Component Signal Transduction, Hoch et al., eds., American Society of Microbiologist, Washington D.C., pp. 431-435, 1995; Parsek and Greenberg, Acyl-homoserine lactone quorum sensing in Gram-negative bacteria: a signaling mechanism involved in associations with higher organisms. Proc Natl Acad Sci USA, 97(16):8789-93, 2000, which are both incorporated by reference herein for their teachings of quorum sensing and acyl homoserine lactone analogs).

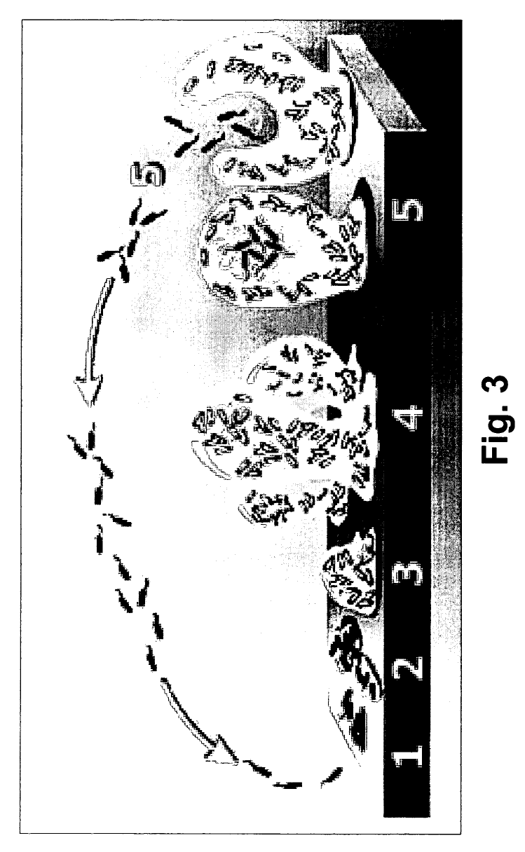

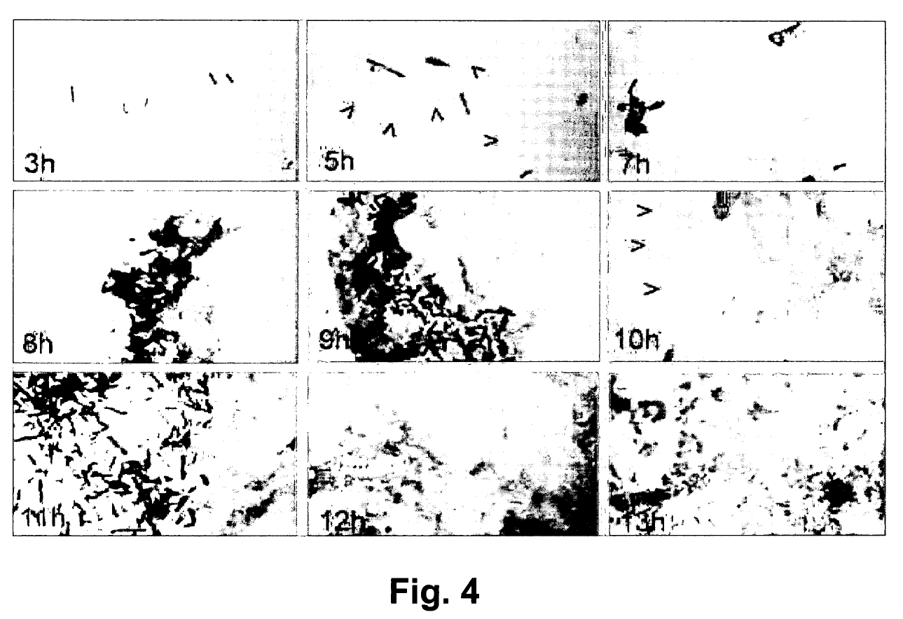

The formation of biofilms is illustrated in FIG. 3. In the early stages of biofilm formation, the biofilm is comprised of a cell layer attached to a surface. The cells grow and divide, forming a dense mat numerous layers thick. These bacteria use quorum sensing to signal each other to reorganize, thereby forming an array of pillars and irregular surface structures. These structures are connected by convoluted channels that deliver food and remove waste. Also, the cells produce a glycocalyx matrix shielding them from the environment and preventing sanitizers from killing them. Microphotographs taken over time show the formation of biofilms in FIG. 4.

It has been demonstrated that quorum sensing signaling mechanisms can be disabled by furanone compounds. Manefield, et al., reported that the marine alga Delisea pulchra produces halogenated furanones that are known to block homoserine lactone activity (FEMS Microbio Lett, 205(1):131-138, 2001, which is incorporated by reference herein for its teachings of Delisea pulchra, its extracts, and furanones). Halogenated acyl furanones have also been shown to act as blockers to homoserine lactone cognate receptor proteins (U.S. Pat. No. 6,455,031 to Davies, et al., which is incorporated by reference herein for its teachings of halogenated furanones).

Disclosed herein are compositions comprising furanones and methods of using such compositions to treat, prevent, inhibit, and/or reduce biofilm formation and/or to disrupt, reduce, or break-down existing biofilms. These compositions and methods can enable more effective disinfection of surfaces, such as food-processing equipment surfaces. While not wishing to be bound by theory, the furanones are believed to be antagonists of acyl-homoserine lactones, inhibiting quorum sensing and the ability of bacterial to form biofilms. Also, furanones are believed to bind with or inhibit bacterial lipopolysaccharide (biofilm or glycocalyx) formation. Thus, the furanones are believed to disrupt or break-down the glyxocalyx matrix of an existing bacterial biofilm as well as prevent the formation of biofilms.

In one aspect, the furanones disclosed herein can be prepared from or obtained by methods described below. In one aspect disclosed herein, furanones can be obtained from the metabolic products of bacterial fermentation. For example, compositions comprising furanones can be obtained from a fermentable substrate comprising one or more fermentive bacteria. In one specific example, milk products comprising Lactobacillus acidophilus can be fermented to provide metabolic products comprising furanones. Such products can be obtained commercially from acidophilus milk. Further, Lactobacillus produces organic acids (lactic acid), lactoperoxidase (peroxide compounds), and bacteriocins (bacterial antibiotics such as nisin, lactacin A-F, and sakacin A, as is discussed below), which can also reduce, inhibit, and/or prevent biofilms. Other fermentative bacterium, such as Lactococcus species and Pediococcus species can be used alone or in combination with other fermentive bacterium to produce the compositions disclosed herein.

Elimination of pathogenic, indicator, and spoilage bacteria on surfaces (e.g., equipment surfaces) prior to forming a biofilm or after a biofilm has been disrupted can be accomplished using a variety of metabolic products of bacterial fermentation. Bacteriocins are one class of metabolic products that have been shown to be effective for killing pathogenic, indicator, and spoilage populations of bacteria. Bacteriocins are antimicrobial proteins produced by bacteria that give bacteria competitive advantage over other species in a particular microenvironment.

Hoover reported that bacteriocins from lactic acid bacteria have the following advantages: the U.S. Food and Drug Administration has approved nisin (a bacteriocin) as a GRAS substance for foods; consumers are resistant to the use of traditional chemical sanitizers; and bacteriocins produced by starter cultures have been used for years as a preservative for fermented foods, such as yogurt and cheese (Microorganisms and their products in the preservation of foods. In: The Microbiological Safety and Quality of Food. Vol. 1. Aspen Publishers, Gaithersburg, et al., eds. 2000, which is incorporated by reference herein for its teaching of bacteriocins and methods of obtaining bacteriocins). Yogurt, cheese, and sausage fermentation starter culture bacteria include species such as Lactobacillus acidophilus, Lactobacillus sakei, Lactococcus lactis subspecies lactis, and Pediococcus aciditactici.

As an example, noted above, Lactobacillus acidophilus can produce organic acids (lactic acid), lactoperoxidase, bacteriocins such as nisin (proven effective and safe for foods for over 50 years), and lactacin A-F among many others (Hoover, Microorganisms and their products in the preservation of foods. In: The Microbiological Safety and Quality of Food. Vol. 1. Aspen Publishers, Gaithersburg, et al., eds. 2000).

In another example, the fermative bacterium Lactobacillus sakei can produce the bacteriocin sakacin A, which has been shown to be effective for killing Listeria populations.

The fermentive bacterium Lactococcus lactis subsp. lactis can produce the bacteriocin nisin, which inhibits Listeria, Staphylococcus, Clostridium, and Bacillus as well as yeast and mold growth. L. lactis subsp. lactis can also produce lacticin (similar to L. acidophilus), which is a hydrophobic polypeptide related to streptococcin from Streptococcus pyogenes. This bacteriocin is effective against Clostridium tyrobutyrieum and is heat stable. L. lactis subsp. lactis can also produce lactostrepticin bacteriocins.

Pediococcus acidilactici is a fermentive bacterium that has traditionally been used to ferment sausages. This bacterium produces the bacteriocin pediocin AcH that inhibits Listeria, Enterococcus, Proprionibacterium, Staphylococcus, Clostridium, and Bacillus. While not wishing to be bound by theory, it is believed that the mechanism of action for pediocin is that it weakens the membranes of vegetative cells and prevents growth after spore germination. A mixture of dried powder of metabolic products of P. acidilaetici grown in nonfat dry milk was able to prevent the growth of Listeria monocytogenes in cottage cheese, half and half cream, and cheddar cheese soup for two weeks at 40° C. (Hoover, Microorganisms and their products in the preservation of foods. In: The Microbiological Safety and Quality of Food. Vol. 1. Aspen Publishers, Gaithersburg, et al., eds. 2000.) Research has also demonstrated that L. monocytogenes, applied to sterilized lean beef, was reduced by applying extract from P. acidilactici. The bacteriocins produced by P. acidilactici were found to be effective on the surface of meat for more than one month of storage.

Fermentive bacteria can be used in the methods disclosed herein. By “fermentive bacteria” is meant any bacteria or combination of bacteria that can enzymatically transform an organic compound (e.g., a carbohydrate). In general, any fermentative bacteria that can produce furanones and/or bacteriocins can be used herein. Various species of fermentive bacteria, suitable for the disclosed methods, are used for fermentation of foods such as yogurt, cheese, sausages, and sauerkraut. These bacteria can produce metabolic products such as furanones, bacteriocins, lactoperoxidase, and organic acids that inhibit the multiplication of other, more dangerous bacteria, such as Listeria monocytogenes. Further, the red algae Delisea pulchra can produce furanone compounds that are able to prevent quorum sensing and thus are able to prevent the formation of biofilms on, for example, food processing equipment (Manefield, et al., FEMS Microbio Lett, 205(1):131-138, 2001). In one aspect, the extract of Delisea pulchra can be used in combination with the metabolic products of fermentive bacteria in the compositions and methods disclosed herein.

In one aspect, disclosed herein are compositions comprising a cell-free fermentate. By “cell-free” is meant that the fermentate is substantially free of cells, typically containing less than about 10⁵ cells/mL fermentate, less than about 10⁴ cells/mL fermentate, less than about 10³ cells/mL fermentate, less than about 10² cells/mL fermentate, or less than about 10 cells/mL fermentate. The compositions disclosed herein can include, for example, one or more furanones and/or one or more bacteriocins. Lactoperoxidase and/or organic acids can also be present in the disclosed compositions.

The disclosed cell-free fermentates can be from one or more fermentive bacteria. For example, the disclosed cell-free fermentates can be prepared by incubating one or more fermentable substrates and one or more fermentive bacteria. Fermentation takes place during the incubation, thereby producing a fermentate. As noted, suitable fermentive bacteria can be any bacteria or combination of bacteria that can enzymatically transform an organic compound (e.g., a carbohydrate). Examples of fermentive bacteria can include, but are not limited to, Lactobacillus species, Lactococcus species, and Pediococcus species. In one aspect, the fermentive bacteria can be one or more bacteria chosen from Lactobacillus acidophilus (e.g., ATCC # 4356), Lactobacillus sakei (e.g., ATCC # 15521), Lactococcus lactis (e.g., ATCC # 11955), and Pediococcus acidilactici (e.g., ATCC # 25742). These bacteria can be particularly useful in the methods and compositions disclosed herein because they can be food safe (i.e., safe to use in, on, or near foods). Also, these bacteria can produce bacteriocins that are specific for Listeria.

In another aspect, it is contemplated that genetically-engineered organisms can be used in the methods disclosed herein. Genetically engineered bacteria can be another kind of fermentive bacteria suitable for use herein. For example, genes that encode one or more bacteriocins and/or one or more furanones can be inserted into an organism. Such engineered organisms can then be used to ferment or produce fermentate containing one or more bacteriocins and/or furanones, which can be isolated and used to treat, prevent, inhibit, reduce, and/or break-down biofilms.

The bacteria can be used separately or collectively in the methods disclosed herein. The cell-free fermentate can be prepared from any single species of fermentive bacteria. For example, the cell-free fermentate can be prepared from species of Lactobacillus alone (e.g., Lactobacillus acidophilus alone or Lactobacillus sakei alone), a species of Lactococcus alone (e.g.,Lactococcus lactis subsp. lactis alone), or a species of Pediococcus alone (e.g., Pediococcus acidilactici alone). In another aspect, the cell-free fermentate can be prepared from any combination of fermentive bacteria. For example, the cell-free fermentate can be prepared from any combination of Lactobacillus species, Lactococcus species, or Pediococcus species (e.g., any combination of Lactobacillus acidophilus, Lactobacillus sakei, Lactococcus lactis subsp. lactis, or Pediococcus acidilactici). In still another aspect, the cell-free fermentate can be prepared by mixing, in any combination, the fermentates and/or cell-free fermentates from any fermentive bacteria or a combination of fermentive bacteria. For example, the cell-free fermentate can be prepared by mixing, in any combination, cell-free fermentates obtained from Lactobacillus species, Lactococcus species, or Pediococcus species (e.g., Lactobacillus acidophilus, Lactobacillus sakei, Lactococcus lactis subsp. lactis, and/or Pediococcus acidilactici), either alone or in combination.

The incubation can take place with, on, or in any one or more fermentable substrate. A fermentable substrate is a material that contains an organic compound such as a carbohydrate that can be transformed (i.e., converted into another compound) by the enzymatic action of a fermentive bacterium. Examples of fermentable substrates include, but are not limited to, non-fat dry milk, vegetables (e.g., corn potatoes, cabbage), starch, grains (e.g., rice, wheat, barley, hops), fruit (e.g., grapes, apples, oranges), sugar, sugarcane, meat (e.g., beef, poultry, pork, sausage), combinations thereof, and the like. Any material that is fermentable can be used as fermentable substrate in the methods disclosed herein. In one aspect, the fermentable substrate is milk or a milk product (i.e., non-fat dry milk), which is commercially available and food safe.

The incubation can take place for any suitable time. For example, the incubation can take place for from about 1 to about 36 hours (h), from about 5 to about 25 h, or from about 10 to about 20 h. In one aspect the incubation can take place for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 h, where any of the stated values can form an upper or lower endpoint when appropriate. In another aspect, the time for incubation can be greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 h. In yet another aspect, the time for incubation can be less than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 h. In still another aspect, the incubation can occur for about 18 h.

The temperature of incubation can be any suitable temperature; typically a temperature suitable for fermentation by the fermentive bacteria. For example, the temperature of incubation can be from about 10 to about 55° C., from about 15 to about 50° C., from about 20 to about 45° C., from about 25 to about 40° C., or from about 30 to about 35° C. In another aspect, the incubation can take place at a temperature of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C., where any of the stated values can form an upper or lower endpoint when appropriate. In still another aspect, the incubation can take place at a temperature greater than or equal to about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C. In yet another aspect, the incubation can take place at a temperature less than or equal to about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55° C. In a further aspect, the incubation can occur at about 37° C.

As noted, the disclosed cell-free fermentates can be prepared by incubating one or more fermentable substrates and one or more fermentive bacteria, resulting in a fermentate. The fermentate can comprise one or more metabolic products (e.g., bacteriocins and/or furanones) as well as other components such as particulate matter, solids, fermentable substrate that has not been fermented, fermentable substrate that has been fermented, fermentive bacteria, debris, media, live and dead cells, cell waste, etc. The metabolic products can be used in the compositions and methods disclosed herein. The metabolic products can be separated or isolated from one or more other fermentate components such as particulate matter, solids, debris, cells, etc. In one aspect, one or more cells are separated from the fermentate, providing a cell-free fermentate.

Any method can be used to separate one or more cells from the fermentate, and thereby provide a cell-free fermentate containing one or more metabolic products. The particular method of separation can depend on, for example, the type and amount of fermentable substrate used, the particular fermentive bacteria used, and the like. In one aspect, one or more cells can be separated from the fermentate by centrifuging and/or filtering. For example, the fermentate can be filtered (one or several times in a multistep process) to remove such components as particulate matter, cells, and the like. The resulting cell-free fermentate can comprise one or more metabolic products. Another method of separating components such as one or more cells from the fermentate is to centrifuge the fermentate, thus producing a supernatant. Depending on the speed and duration of the centrifugation, the supernatant can be cell free (i.e., the cell-free fermentate) or the supernatant can contain, et al., cells, which can be filtered or further centrifuged to provide a cell-free fermentate.

Centrifugation is well known in the art. In one aspect, the centrifugation can take place in a Sorvall SS-34 rotor. The speed of centrifugation can be at, for example, about 5,000 rpm, 10,000 rpm, 15,000 rpm, 20,000 rpm, 25,000 rpm, or 30,000 rpm. In one aspect, the speed of the centrifugation can be at least about 5,000 rpm. The time of centrifugation can be from about 5 minutes to 1 h, from about 10 minutes to about 45 minutes, or about 30 minutes. In one aspect, the time of the centrifugation is at least about 10 minutes, or at least about 15 minutes.

In one aspect, one or more cells can be separated from the fermentate (e.g., after centrifugation), by filtration. Various filters can be used to filter the fermentate or a supernatant containing cells. For example, a microfilter with a pore size of from about 0.01 to about 1 μm, from about 0.05 to about 0.5 μm, or from about 0.1 to about 0.2 μm. In another aspect, the filter can have a pore size of about 0.01, 0.02, 0.03, 0.04, 0.05, 0.06, 0.07, 0.08, 0.09, 0.1, 0.2, 0.3, 0.4, 0.5, 0.6, 0.8, 0.9, or 1 μm, where any of the stated values can form an upper or lower endpoint when appropriate. In yet another aspect, the filter can have a pore size of greater than or equal to about 0.01, 0.02, 0.03, 0.04, 0.05, 0.06, 0.07, 0.08, 0.09, 0.1, 0.2, 0.3, 0.4, 0.5, 0.6, 0.8, 0.9, or 1 μm. In still another aspect, the filter can have a pore size of less than or equal to about 0.01, 0.02, 0.03, 0.04, 0.05, 0.06, 0.07, 0.08, 0.09, 0.1, 0.2, 0.3, 0.4, 0.5, 0.6, 0.8, 0.9, or 1 μm. In a further aspect, the filter can have a pore size of about 0.2 μm, such as is available from Millipore (Billerica, Mass.). The fermentate can, in one aspect, be filtered with a sterilizing filter.

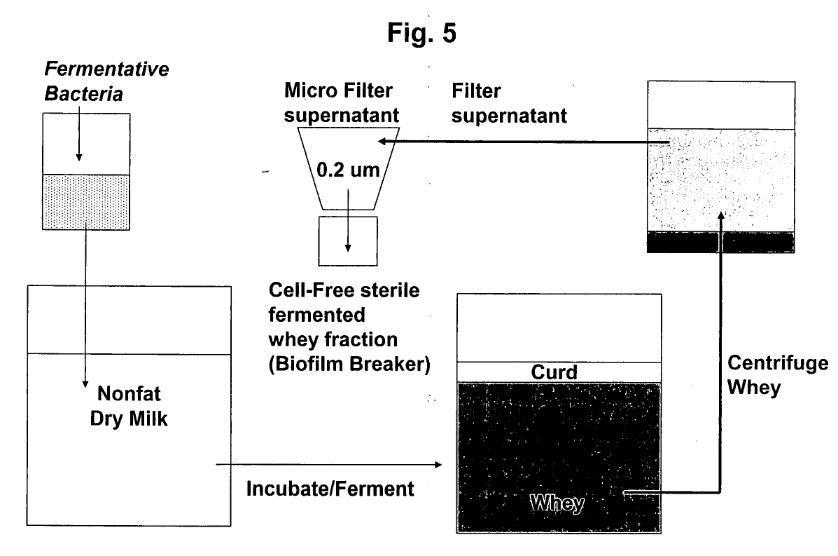

One example of a method for preparing a cell-free fermentate is shown in FIG. 5. In this example, a fermentable substrate such as nonfat-dry milk is fermented using fermentive bacteria. The fermentive bacteria can be, for example, one or more bacteria chosen from Lactobacillus acidophilus, Lactobacillus sakei, Lactococcus lactis subsp. lactis, and/or Pediococcus acidilactici, used separately or collectively. The fermentation results in a fermentate comprising a curd fraction and whey fraction. Cells can be separated from the fermentate by collecting the whey fraction (e.g., separating the whey fraction from the curd fraction), centrifuging, and filtering the resulting supernatant using a sterilizing filter (0.2 μm). Alternatively, the fermentate (e.g., the whey fraction and the curd fraction) can be centrifuged, and the resulting supernatant can be filtered. The resulting cell-free fermentate can be used to treat surfaces on, for example, food processing equipment. These compositions can contain bacteriocins, peroxidases (e.g., lactoperoxidases), organic acids, and furanones. These compositions can be effective for preventing biofilm formation, reducing, breaking-down, or eliminating already formed biofilms, and/or for killing pathogenic, indicator, and spoilage bacteria associated with food processing equipment and various food types.

In one aspect, an extract from Delisea pulchra can be added to the cell-free fermentate. Extract from Delisea pulchra can be obtained from any method known in the art and can include highly purified or crude extract. In one aspect, the extract from Delisea pulchra can be obtained by the method disclosed in Manefield, et al., FEMS Microbio Lett, 205(1):131-138, 2001, which is incorporated by reference herein for its teachings of Delisea pulchra and its extracts.

Depending on the intended mode of administration, some of which are discussed below, the compositions disclosed herein can be in the form of solid, semi-solid, liquid, or gel forms, such as, for example, tablets, pills, capsules, powders, liquids, suspensions, dispersions, or emulsions. Also, the compositions disclosed herein can be in a form suitable for dilution. That is, the compositions can be in the form of an aqueous or non-aqueous stock solution, concentrate, concentrated solution, dispersion, emulsion, or suspension that can be diluted to a desired concentration with a suitable solvent. Similarly, the compositions can be in the form of a powder, paste, cream, or solid that can be reconstituted or mixed with a solvent and diluted to a desired concentration to form a solution or dispersion, emulsion, or suspension.

The compositions disclosed herein can, in one aspect, further comprise one or more additional components, e.g., carrier, adjuvant, solubilizing agent, suspending agent, diluent, and/or consumer acceptable agent. By “consumer acceptable agent” is meant a material that is not biologically or otherwise undesirable when consumed, e.g., an agent that is acceptable when used in or on foods and beverages and which can be consumed by an individual (e.g., human, pet, livestock, etc.) along with the selected active components without causing significant undesirable biological effects or interacting in a deleterious manner with any of the other components of the composition in which it is contained. For example, a consumer acceptable agent can be any compound generally recognized as safe (GRAS).

The compositions disclosed herein can further comprise a carrier. The term “carrier” means a compound, composition, substance, or structure that, when in combination with a compound or composition disclosed herein, aids or facilitates preparation, storage, administration, delivery, effectiveness, selectivity, or any other feature of the compound or composition for its intended use or purpose. For example, a carrier can be selected to minimize any degradation of the active components and to minimize any adverse side effects. Examples of suitable aqueous and non-aqueous carriers, diluents, solvents include water, ethanol, polyols (propyleneglycol, polyethyleneglycol, glycerol, and the like), vegetable oils, and suitable mixtures thereof.

The compositions disclosed herein can also comprise adjuvants such as preserving, wetting, emulsifying, suspending agents, and dispensing agents. Prevention of the action of other microorganisms can be ensured by various antifungal agents, for example, parabens, chlorobutanol, phenol, sorbic acid, and the like. It may also be desirable to include surfactants, binders, as for example, carboxymethylcellulose, alignates, gelatin, polyvinylpyrrolidone, sucrose, and acacia, humectants, as for example, glycerol, wetting agents, as for example, cetyl alcohol, and glycerol monostearate, adsorbents, as for example, kaolin and bentonite, and lubricants, as for example, talc, calcium stearate, magnesium stearate, solid polyethylene glycols, sodium lauryl sulfate, or mixtures thereof.

Suitable suspending agents can include, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

The disclosed compositions can also comprise solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

The disclosed compositions can also comprise perfuming agents and/or fragrances.

The compositions disclosed herein can be applied to surfaces in any manner known in the art. For example, the compositions disclosed herein can be poured, sprayed, misted, wiped, or mopped onto a surface. In another example, a surface can be immersed, dipped, or soaked into the compositions disclosed herein. In still another example, the compositions disclosed herein can be dispersed as fine particulates or a gas by, for example, a fogging system.

In one aspect, the disclosed compositions can be contacted to a surface using an electrostatic sprayer. An electrostatic sprayer can coat substantially all surfaces while requiring a minimal amount of material. Typical spraying methods for applying sanitizers in food processing facilities can be problematic because of the volume of sanitizers that must be used and the inability of these systems to adequately coat joints, cracks, and crevices with sanitizer. Electrostatic spraying was developed over two decades ago and is used to apply pesticides to row crops. Law (Embedded-electrode electrostatic induction spray charging nozzle: theoretical and engineering design. Transact of the ASAE, 12:1096-1104, 1978, which is incorporated herein by reference for its teachings of electrostatic spraying) developed an electrostatic spray-charging system using air atomization, which has been used to achieve a 7-fold increase in spray deposition over conventional application methods. In a later study, Law, et al., reported a 1.6 to 24-fold increase in deposition (Law and Lane, Electrostatic deposition of pesticide spray onto foliar targets of varying morphology. Transact of the ASAE, 24:1441-1448, 1981, which is incorporated herein by reference for its teachings of electrostatic spraying).

Herzog, et al., demonstrated that insect control on cotton plants was equal to or better than conventional spray application using only one-half the amount of insecticide (Herzog, et al., Evaluation of an electrostatic spray application system for control of insect pests in cotton. J Econ Entomol, 6:637-640, 1983, which is incorporated herein by reference for its teachings of electrostatic spraying).

It has been shown in laboratory studies that conventional methods for spraying chicken carcasses required about 5 ozs. (about 148 mL) of sanitizer in order to be effective; whereas, using electrostatic spraying, only about 0.3 ozs. (about 9 mL) was generally required. Of course, the amount of the compositions disclosed herein will depend on the surface area to be treated, the composition concentration, and the like. The amount of the disclosed compositions can be determined by one of skill in the art.

Electrostatic spraying of the cell-free fermentate of the species of fermentive bacteria listed previously, optionally in combination with the red algae extract of Delisea pulchra, can be used as a means of applying this composition to equipment surfaces and foods, thus preventing biofilm formation, eliminating already formed biofilms, and killing pathogenic, indicator, and spoilage bacteria. Application of the disclosed compositions using electrostatic spraying or an alternative fogging system can significantly increase deposition and decrease the amount of product necessary to prevent biofilms and breakdown already formed biofilms. While not wishing to be bound by theory, this is believed to be due to the fact that food processing equipment surfaces, and meat, poultry and vegetable surfaces, have a native positive charge. As high-pressure air and sanitizer are forced through a small aperture in the electrostatic spray nozzle, the air shears the sanitizer into tiny droplets (approximately 30 micrometers in diameter). These droplets are then exposed to an electrical charge as they exit the nozzle head. This transfers a negative charge to the sanitizer particle, which then has a particular affinity for the surfaces in the area, such as processing equipment. Because the deposition of sanitizer to the surface being treated can be much more efficient with electrostatic spraying, much less sanitizer can be used to result in the same bacterial disinfection rate when compared to commonly-used commercial loggers or sprayers.

In one aspect, disclosed herein are methods for preventing biofilm formation, breaking-down or reducing existing biofilms, and/or reducing a population of bacteria, for example pathogenic, indicator, and spoilage bacteria by contacting (e.g., by electrostatic spraying) a surface with the compositions (e.g., cell free fermentate) disclosed herein. In another aspect, disclosed herein are methods of preventing the transfer of pathogenic, indicator, and spoilage bacteria from biofilms on food processing equipment and surfaces to uncontaminated, ready-to-eat products by contacting a surface with the disclosed compositions. By preventing biofilm formation, breaking-down existing biofilms, and/or reducing bacterial populations, the compositions and methods disclosed herein can have a positive impact on preventing contamination of fully-cooked, ready-to-eat meat and poultry products with bacteria such as Listeria monocytogenes that commonly forms biofilms on processing equipment, in coolers, and in freezers. Further, the disclosed compositions and methods can have a beneficial impact on the safety of ready-to-eat foods and vegetables. Also disclosed are methods for increasing the shelf-life of fresh foods such as meat, poultry, fruit, vegetables, seafood, and milk by contacting a surface with the disclosed compositions. The disclosed compositions can also be used on many foods to decrease pathogenic bacteria on the surface of the food and to prevent their growth (e.g., Listeria on hot dogs or E. coli O157:H7 on beef carcasses.

Prevention of biofilm formation and breakdown of already formed biofilms can greatly decrease post-processing contamination of fully-cooked, ready-to-eat meat products, vegetables, food processing equipment surfaces, coolers, freezers, and food contact surfaces with regard to the level of contamination with pathogenic, indicator, and spoilage bacterial populations and can greatly enhance the efficacy of commercially used sanitizers.

In one aspect, disclosed herein are methods of treating a surface by contacting (e.g., electrostatic spraying) the surface with an effective amount of a composition disclosed herein. The term “effective amount” means that the amount of the composition used is of sufficient quantity to provide the desired result (e.g., reduction or prevention of biofilms). As will be pointed out below, the exact amount required will vary from application to application, depending on the type, age, and general condition of the biofilm, the particular composition used, its mode of administration, the type and scale of the surfaces being treated, and the like. Thus, it is not possible to specify an exact “effective amount.” However, an appropriate effective amount can be determined by one of ordinary skill in the art using only routine experimentation.

Any surface can be treated by the methods disclosed herein. Examples of types of surfaces that can be treated by the methods disclosed herein include, but are not limited to, food processing equipment surfaces such as tanks, conveyors, floors, drains, coolers, freezers, equipment surfaces, walls, valves, belts, pipes, joints, crevasses, combinations thereof, and the like. The surfaces can be metal, for example, aluminum, steel, stainless steel, chrome, titanium, iron, alloys thereof, and the like. The surfaces can also be plastic, for example, polyolefins (e.g., polyethylene, polypropylene, polystyrene, poly(meth)acrylate, acrylonitrile, butadiene, ABS, acrylonitrile butadiene, etc.), polyester (e.g., polyethylene terephthalate, etc.), and polyamide (e.g., nylon), combinations thereof, and the like. The surfaces can also be brick, tile, ceramic, porcelain, wood, vinyl, linoleum, or carpet, combinations thereof, and the like. The surfaces can also, in other aspects, be food, for example, beef, poultry, pork, vegetables, fruits, seafood, combinations thereof, and the like.

Also disclosed are systems comprising a surface (e.g., food processing equipment surface) and a composition disclosed herein.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

The purpose of these studies was to determine if the sterile, cell-free fermentates of Pediococcus acidilactici, Lactococcus lactis subsp. lactis, Lactobacillus acidophilus, and Lactobacillus sakei were able to 1) coat a surface to prevent surface biofilm formation by Listeria monocytogenes (LM), 2) prevent the attachment of LM to a surface, 3) prevent biofilm formation by LM in an aqueous environment, and 4) remove or break-up already formed biofilms of LM.

### Example 1

### Cell-Free Fermentate

The cell-free fermentates of four bacteria were created as shown in FIG. 5. The bacterial species used to create the fermentates were Lactococcus lactis subsp. lactis (ATCC # 11955), Pediococcus acidilactici (ATCC # 25742), Lactobacillus acidophilus (ATCC # 4356), and Lactobacillus sakei (ATCC # 15521). Cultures of each bacterium were placed on nonfat-dry milk and incubated for 18 h at 37° C. After incubation the whey fraction was separated from the curd fraction. The whey was then centrifuged for 10 min at 25,000 rpm. The whey was then filtered using a sterilizing filter (0.2 μm pore size from Millipore, Billerica, Mass.). This resulted in a sterile, cell-free fermentate.

### Example 2

### Controls

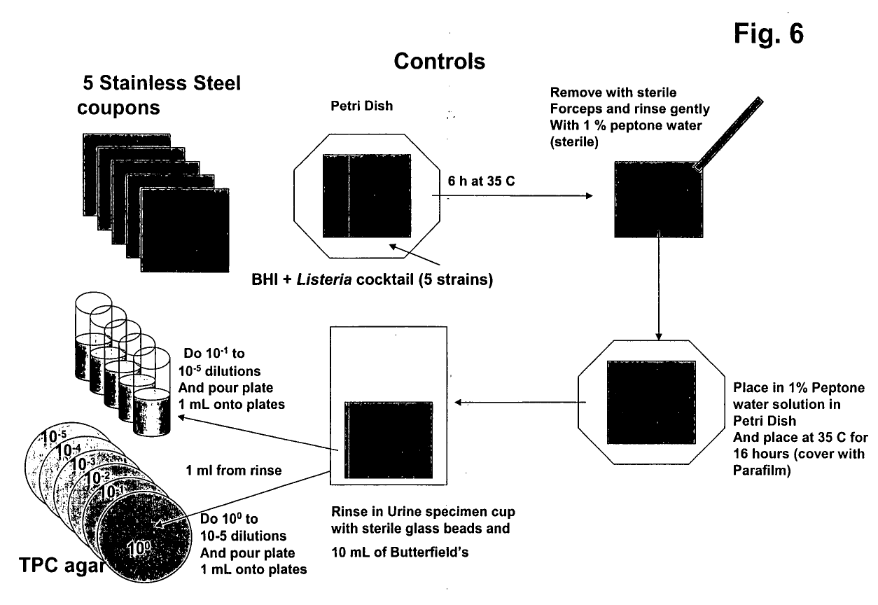

This example is shown schematically in FIG. 6. Five stainless steel coupons (1 in²; 6.5 cm²) were placed into a sterile Petri dish and sterile brain heart infusion (BHI) broth and LM were added to the dish. This was incubated at 35° C. for 6 h to attach the LM to the surface of the coupon. The coupons were removed from the dish with sterile forceps, and rinsed gently with 1% sterile peptone broth. The coupon was then placed into a Petri dish containing 1% sterile peptone broth, covered with Parafilm, and incubated at 35° C. for 16 h to allow the LM biofilm to grow. The coupon was then shaken in a sterile urine specimen cup with sterile glass beads and 10 mL of Butterfield's Phosphate Buffer to remove the biofilm from the coupon. The sample was diluted appropriately, pour plated using total plate count agar, incubated at 35° C. for 24 h and counted.

### Example 3

### Coating Study

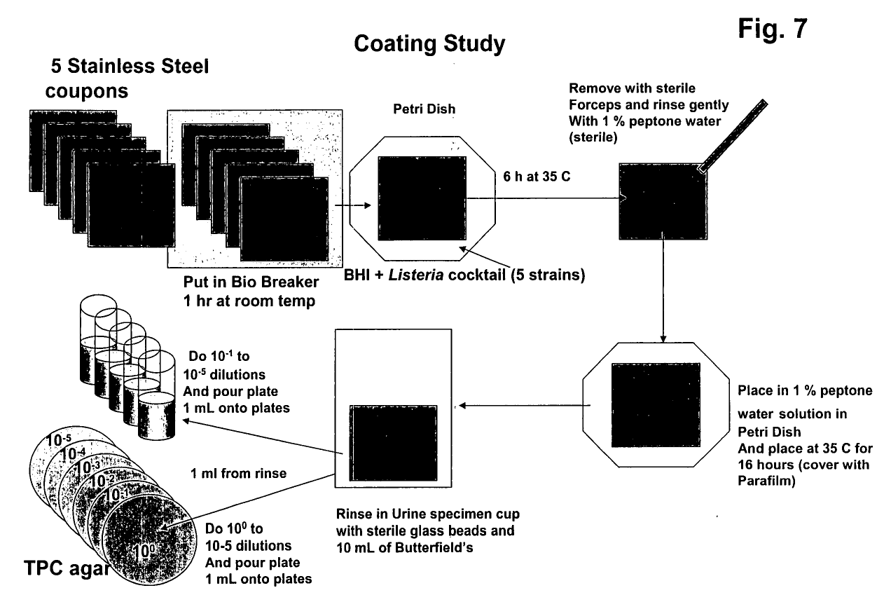

This example is shown schematically in FIG. 7. Five stainless steel coupons (1 in²; 6.5 cm²) were placed into the sterile, cell-free fermentate from Pediococcus acidilactici, Lactococcus lactis subsp. lactis, Lactobacillus acidophilus, and Lactobacillus sakei and allowed to remain for 1 h at room temperature (about 20° C.). The coupon was then placed into a sterile Petri dish and sterile brain heart infusion (BHI) broth and LM were added to the dish. This was incubated at 35° C. for 6 h to attach the LM to the surface of the coupon. The coupons were removed from the dish with sterile forceps, and rinsed gently with 1% sterile peptone broth. The coupon was then placed into a Petri dish containing 1% sterile peptone broth, covered with Parafilm, and incubated at 35° C. for 16 h to allow the LM biofilm to grow. The coupon was then shaken in a sterile urine specimen cup with sterile glass beads and 10 mL of Butterfield's Phosphate Buffer to remove the biofilm from the coupon. The sample was diluted appropriately, pour plated using total plate count agar, incubated at 35° C. for 24 h and counted.

### Example 4

### Pre-Attachment Study

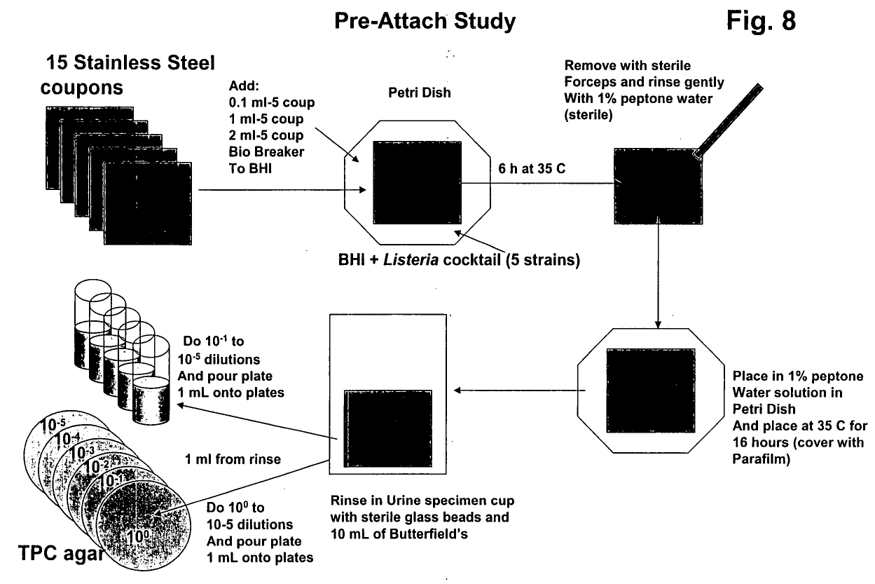

This example is shown schematically in FIG. 8. Five stainless steel coupons (1 in²; 6.5 cm²) were placed into a sterile Petri dish with sterile, cell-free fermentate from Pediococcus acidilactici, Lactococcus lactis subsp. lactis, Lactobacillus acidophilus, and Lactobacillus sakei sterile brain heart infusion (BHI) broth, and LM This was incubated at 35° C. for 6 h to determine if the LM would be able to attach to the surface of the coupon. The coupons were removed from the dish with sterile forceps, and rinsed gently with 1% sterile peptone broth. The coupon was then placed into a Petri dish containing 1% sterile peptone broth, covered with Parafilm, and incubated at 35° C. for 16 h to allow the LM biofilm to grow. The coupon was then shaken in a sterile urine specimen cup with sterile glass beads and 10 mL of Butterfield's Phosphate Buffer to remove the biofilm from the coupon. The sample was diluted appropriately, pour plated using total plate count agar, incubated at 35° C. for 24 h and counted.

### Example 5

### Pre-Biofilm Study

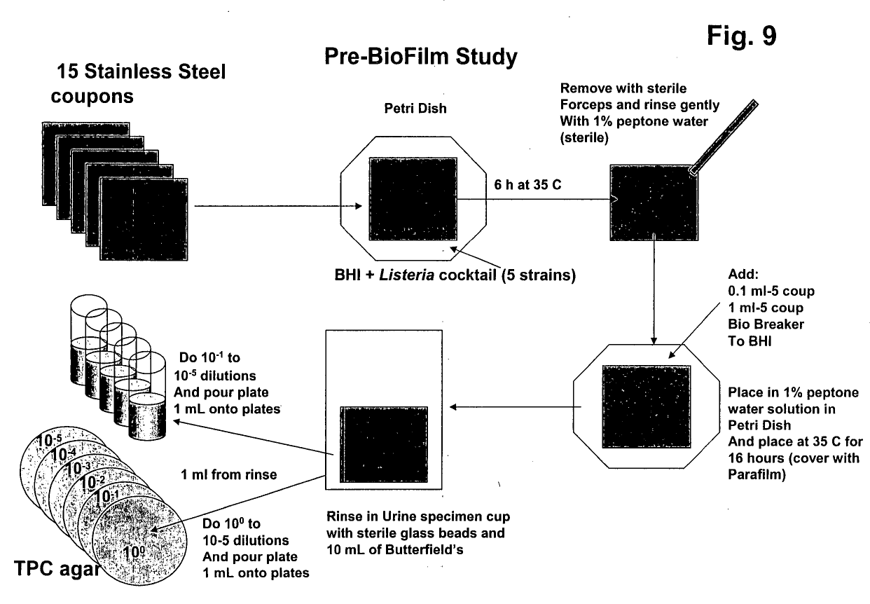

This example is shown schematically in FIG. 9. Five stainless steel coupons (1 in²; 6.5 cm²) were placed into a sterile Petri dish and sterile brain heart infusion (BHI) broth and LM were added to the dish. This was incubated at 35° C. for 6 h to attach the LM to the surface of the coupon. The coupons were removed from the dish with sterile forceps, and rinsed gently with 1% sterile peptone broth. The coupon was then placed into a Petri dish containing 1% sterile peptone broth, sterile, cell-free fermentate from Pediococcus acidilactici, Lactococcus lactis subsp. lactis, Lactobacillus acidophilus, and Lactobacillus sakei, covered with Parafilm, and incubated at 35° C. for 16 h to determine if the LM biofilm was able to grow. The coupon was then shaken in a sterile urine specimen cup with sterile glass beads and 10 mL of Butterfield's Phosphate Buffer to remove the biofilm from the coupon. The sample was diluted appropriately, pour plated using total plate count agar, incubated at 35° C. for 24 h and counted.

### Example 6

### Post-Biofilm Study

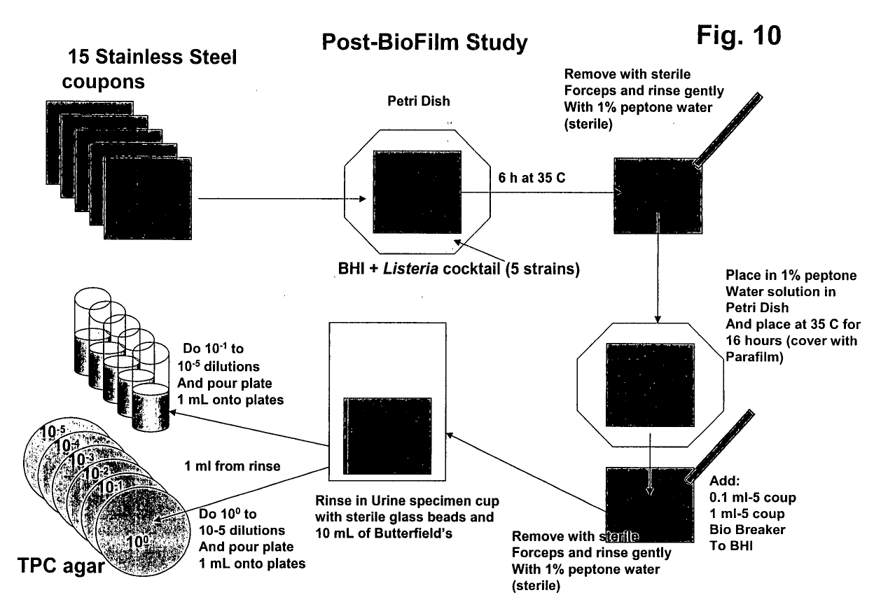

This example is shown schematically in FIG. 10. Five stainless steel coupons (1 in²; 6.5 cm²) were placed into a sterile Petri dish and sterile brain heart infusion (BHI) broth and LM were added to the dish. This was incubated at 35° C. for 6 h to attach the LM to the surface of the coupon. The coupons were removed from the dish with sterile forceps, and rinsed gently with 1% sterile peptone broth. The coupon was then placed into a Petri dish containing 1% sterile peptone broth, covered with Parafilm, and incubated at 35° C. for 16 h to allow the LM biofilm to grow. The coupon was then removed and placed into a Petri dish containing sterile, cell-free fermentate from Pediococcus acidilactici, Lactococcus lactis subsp. lactis, Lactobacillus acidophilus, and Lactobacillus sakei to determine if the biofilm could be broken down by the fermentate and shaken in a sterile urine specimen cup with sterile glass beads and 10 mL of Butterfield's Phosphate Buffer to remove the biofilm from the coupon. The sample was diluted appropriately, pour plated using total plate count agar, incubated at 35° C. for 24 h and counted.

Results:

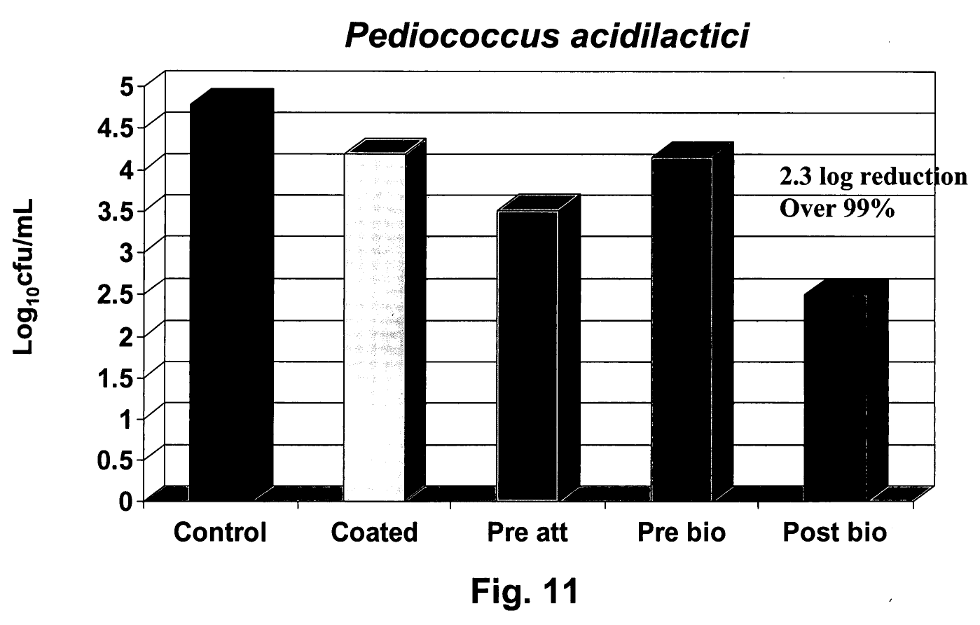

The results of Examples 2-6 are illustrated in FIGS. 11-14.

Cell-free fermentate from Pediococcus acidilactici was able to reduce Listeria monocytogenes before attachment to the coupon by 1.3 logs and was able to kill 2.3 logs (>99%) of LM that was already encased in a biofilm (FIG. 11). These are substantial reductions because chemical sanitizers have been shown to only decrease bacteria in biofilms by approximately 60%.

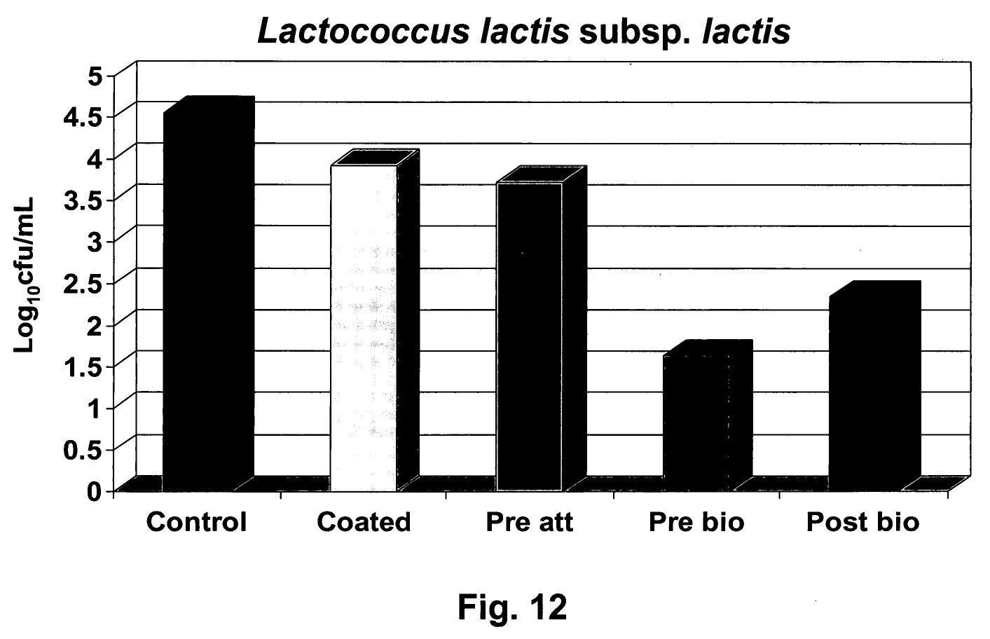

Cell-free fermentate from Lactococcus lactis subsp. lactis was able to reduce Listeria monocytogenes during the biofilm formation period on the coupon by 2.92 logs (almost 99.9%) and was able to kill 2.2 logs (>99%) of LM that was already encased in a biofilm (FIG. 12).

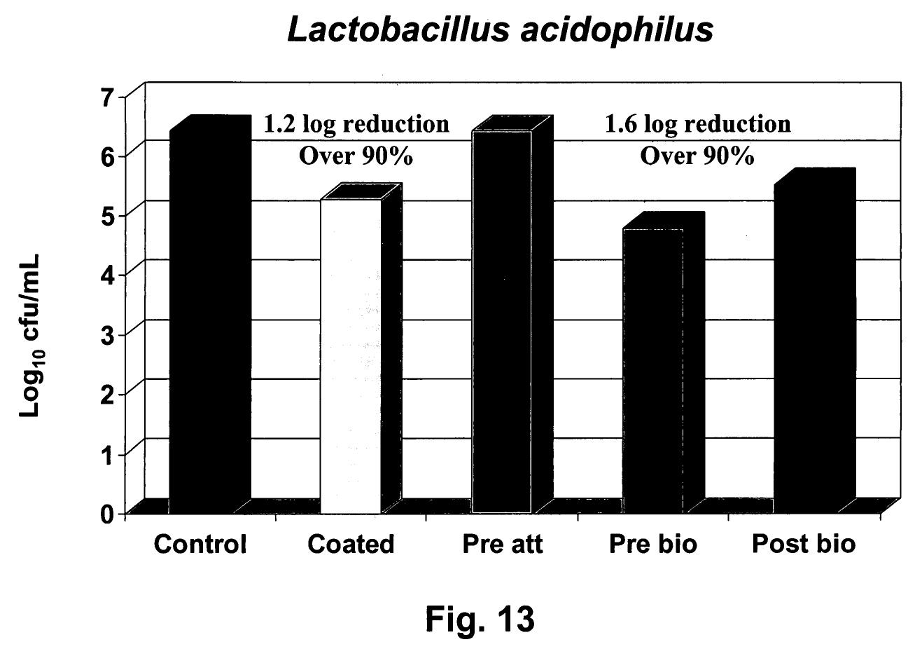

Cell-free fermentate from Lactobacillus acidophilus was able to reduce Listeria monocytogenes by coating the coupon prior to exposure to LM by 1.2 logs (>90%) and was able to reduce LM during the biofilm formation period on the coupon by 1.6 logs (>90%) (FIG. 13).

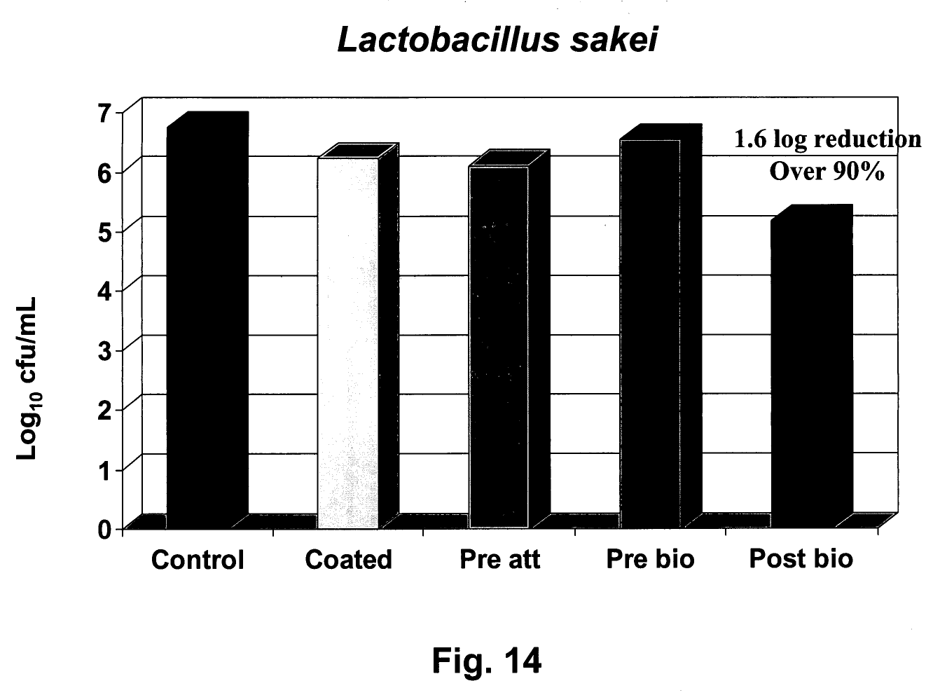

Cell-free fermentate from Lactobacillus sakei was able to reduce Listeria monocytogenes before attachment to the coupon by 0.65 logs and was able to kill 1.6 logs (>90%) of LM that was already encased in a biofllm (FIG. 14).

1. A composition for treating biofilms on a surface, comprising:
one or more cell-free fermentates.

2. The composition of claim 1, wherein the cell-free fermentate is from one or more fermentive bacteria chosen from Lactobacillus species, Lactococcus species, and Pediococcus species.

3. The composition of claim 1, wherein the cell-free fermentate is from one or more fermentive bacteria chosen from Lactobacillus acidophilus, Lactobacillus sakei, Lactococcus lactis subspecies lactis, and Pediococcus acidilactici.

4. The composition of claim 1, further comprising an extract from Delisea pulchra.

5. The composition of claim 4, further comprising one or more compounds chosen from a carrier, diluent, adjuvant, solubilizing agent, and suspending agent.

6. The composition of claim 1, further comprising one or more compounds chosen from a carrier, adjuvant, solubilizing agent, suspending agent, diluent, and consumer acceptable agent.

7. A composition for treating biofilm on a surface, comprising a cell-free fermentate, wherein the cell-free fermentate is prepared by the steps, comprising:
a. incubating one or more fermentable substrates and one or more fermentive bacteria, thereby providing a fermentate comprising one or more cells;
b. separating one or more cells from the fermentate, thereby providing the cell-free fermentate.

8. The composition of claim 7, wherein the fermentable substrate is chosen from a vegetable, starch, grain, fruit, sugar, sugarcane, meat, and non-fat dry milk, or a combination thereof.

9. The composition of claim 7, wherein the fermentable substrate is non-fat dry milk.

10. The composition of claim 7, wherein the fermentive bacteria is one or more bacteria chosen from Lactobacillus species, Lactococcus species, and Pediococcus species.

11. The composition of claim 7, wherein the fermentive bacteria is one or more bacteria chosen from Lactobacillus acidophilus, Lactobacillus sakei, Lactococcus lactis subspecies lactis, and Pediococcus acidilactici.

12. The composition of claim 7, wherein separating is accomplished by centrifuging.

13. The composition of claim 7, wherein separating is accomplished by filtering.

14. The composition of claim 7, wherein the fermentable substrate is non-fat dry milk, and wherein separating is accomplished by collecting a whey fraction, centrifuging the whey fraction, thereby providing a supernatant, and filtering the supernatant.

15. A method of treating a surface, comprising:
contacting a surface with an effective amount of the composition of claim 1.

16. The method of claim 15, wherein there is a biofilm on the surface.

17. The method of claim 16, wherein the biofilm comprises one or more microorganisms chosen from Bacillus, Campylobacter, Clostridium, Enterococcus, Escherichia, Fusarium, Listeria, Proprionibacterium, Pseudomonas, Salmonella, Staphylococcus, Streptococcus, Shewanella, and Toxoplasma species.

18. The method of claim 15, wherein the composition further comprises an extract from Delisea pulchra.

19. The method of claim 15, wherein the surface is a food processing equipment surface.

20. The method of claim 15, wherein the surface is on meat, poultry, pork, vegetable, fruit, or seafood.

21. The method of claim 15, wherein the surface is metal, plastic, brick, tile, ceramic, porcelain, wood, vinyl, linoleum, or carpet.

22. The method of claim 15, wherein contacting is accomplished by electrostatic spraying.

23. A method for preventing the transfer of pathogenic, indicator, or spoilage bacteria from a biofilm on a food processing equipment surface to a food product, comprising spraying with an electrostatic sprayer the composition of claim 1 onto the equipment surface.

24. A method for increasing the shelf-life of a food, comprising contacting the food with the composition of claim 1.

25. A system comprising the composition of claim 1 and a surface.

26. The system of claim 25, wherein the surface is a food processing equipment surface.
